(19) United States
(12) Patent Application Publication
(10) Pub. No.: US 2023/0149731 A1
(43) Pub. Date: May 18, 2023

(54) SLEEP AND SEXUAL WELLNESS DEVICE

(71) Applicant: Lexington International, LLC, Boca Raton, FL (US)

(72) Inventors: David MICHAELS, Boca Raton, FL (US); Sergio D. BORJAS, Boca Raton, FL (US); Randall VELIKY, Lighthouse Point, FL (US); James P. SMERIGLIO, JR., Jupiter, FL (US)

(21) Appl. No.: 17/919,705

(22) PCT Filed: Apr. 23, 2021
(86) PCT No.: PCT/US2021/028883

§ 371 Date: Oct. 18, 2022

Related U.S. Application Data
(60) Provisional application No. 63/015,085, filed on Apr. 24, 2020.

(51) Int. Cl. A61N 5/06; A61M 21/02

(52) U.S. Cl. CPC A61N 5/0618; A61M 21/02; A61N 2005/0645; A61N 2005/0652

(57) ABSTRACT

A device and a method for promoting sleep. The apparatus for promoting sleep may comprise at least one flexible pad configured to contour an area of skin over a target region, the target region including at least one of a group consisting of a carotid artery, a femoral artery, and a perineum, the at least one flexible pad having a plurality of light sources and a controller having processing circuitry in communication with the plurality of light sources, the processing circuitry configured operate the plurality of light sources or a predetermined period of time to increase, decrease or modulate sleep related hormone production in the target region.

### TECHNICAL FIELD

This disclosure relates to an apparatus and method for promoting sleep and sexual wellness.

### INTRODUCTION

There are many different factors which can impact the ability to fall asleep as well as the amount and quality of sleep. The importance of regular sleep and an adequate amount of sleep is well-known and can impact all aspects of daily living as well as an individual's overall health. An insufficient amount of sleep can lead to underperformance at the workplace and impaired performance in any life activity which can pose a danger to oneself as well as others. An insufficient amount of regular sleep for an extended period of time can also increase an individual's risk for diabetes, obesity, depression, high blood pressure, and strokes. Many people find it difficult to obtain an adequate amount of sleep as well as the quality of sleep required to function day-to-day.

There are numerous pharmacological options available on the market that can aid in sleep. These types of medications may be addictive with extended use. These sleep-inducing medications can also be dangerous if combined with other prescription and non-prescription medications, drugs, and/or alcohol. An individual who has taken a sleep-inducing medication may experience morning sleepiness and other side-effects including feeling confused, dizzy, unsteady, or forgetful. The side-effects of pharmacological options that aid with sleep can also worsen as people age. Additionally, if an individual takes a medication to help with sleep, it can be difficult to counteract if the individual taking the medication needs to be roused unexpectedly, for example in the case of an emergency.

It is believed that certain wavelengths of light can help an individual fall asleep more easily and effectively. For example, there is a theory that red light aids in melatonin production and/or modulation (melatonin is a hormone that naturally makes people feel drowsier). One study has found that body irradiation with red light improves the quality of sleep and offers a nonpharmacologic and noninvasive therapy to help with sleep which in turn positively impacts endurance performance in athletes. See e.g., Journal of Athletic Training, “Red Light and the Sleep Quality and Endurance Performance of Chinese Female Basketball Players” by Jiexiu Zhao et. al, November-December 2012, 47(6), pgs. 673-678. Using certain wavelengths of light can help with sleep without the unwanted side-effects that are often seen with various pharmacological options.

### SUMMARY

The present invention relates to an apparatus and method for promoting sleep. In an exemplary embodiment, the apparatus for promoting sleep comprises at least one flexible pad configured to contour an area of skin over a target region, the target region including at least one of a group consisting of a carotid artery, a femoral artery, and a perineum, the at least one flexible pad having a plurality of light sources; and a controller having processing circuitry in communication with the plurality of light sources, the processing circuitry configured to operate the plurality of light sources for a predetermined period of time to increase, decrease or modulate sleep.

In one embodiment, the plurality of light sources is configured to emit at least two wavelengths of light.

In one embodiment, the plurality of light sources is a plurality of light emitting diodes configured to emit light having wavelengths between 300-1300 nanometers.

In one embodiment, the at least one flexible pad is configured to contour an area of skin over a carotid artery target region, the at least one flexible pad including a first portion and a second portion, the first portion sized to contour an area of skin around at least a portion of a user's neck and the second portion sized to contour around at least a portion of at least one of a group consisting of the user's shoulders, upper chest, and upper back.

In one embodiment, the at least one flexible pad is configured to contour an area of skin over a perineum target region, the at least one flexible pad sized to contour around at least one of a group consisting of at least a portion of a user's buttocks and at least a portion of the user's perineum.

In one embodiment, the at least one flexible pad is configured to contour the area of skin over a femoral artery region and outer thigh.

In one embodiment, the at least one flexible pad includes at least three flexible pads.

In one embodiment, the predetermined period of time is at least one minute.

In one embodiment, the plurality of light sources is recessed within the at least one flexible pad.

In another embodiment, a method for promoting sleep comprising positioning at least one flexible pad configured to contour an area of skin over a target region, the target region including at least one of a group consisting of a carotid artery, a femoral artery, and a perineum, includes emitting light for a predetermined period of time from a plurality of light sources, the plurality of light sources being recessed within the at least one flexible pad, and is in communication with a controller having processing circuitry, the processing circuitry configured to cause light from the plurality of light sources to be emitted for a predetermined period of time.

In one embodiment, the plurality of light sources is a plurality of light emitting diodes configured to emit at least two wavelengths of light.

In one embodiment, the predetermined period of time is at least one minute.

In one embodiment, the at least one flexible pad is configured to contour the area of skin over the carotid artery target region, the at least one flexible pad including a first portion and a second portion, the first portion sized to contour the area of skin around at least a portion of a user's neck and the second portion sized to contour around at least a portion of at least one of a group consisting of the user's shoulders, upper chest, and upper back.

In one embodiment, the at least one flexible pad is configured to contour the area of skin over a perineum target region, the at least one flexible pad sized to contour around at least one of a group consisting of at least a portion of a user's buttocks and at least a portion of the user's perineum.

In one embodiment, the at least one flexible pad includes at least three flexible pads.

In one embodiment, the plurality of light sources is configured to emit light having wavelengths between 300-1300 nanometers.

In one embodiment, the at least one flexible pad is configured to contour an area of skin over a femoral artery region and outer thigh.

In one embodiment, the processing circuitry is configured to operate the plurality of light sources to emit a predetermined amount of light for the predetermined period of time.

In one embodiment, the plurality of light sources is configured to emit at least red, near infrared, and infrared light.

In another embodiment, the apparatus for promoting sleep comprises a first flexible pad, a second flexible pad, and a third flexible pad, the first, second and third flexible pads configured to contour an area of skin over a target region, the target region including a carotid artery, a femoral artery, or a perineum, each of the first, second, and third flexible pads having a plurality of light sources recessed within each flexible pad, the first flexible pad configured to contour an area of skin over at least a portion of the carotid artery, the first flexible pad including a first portion and a second portion, the first portion sized to contour the area of skin around at least a portion of a user's neck and the second portion sized to contour around at least a portion of at least one of a group consisting of the user's shoulders, upper chest, and upper back, the second flexible pad configured to contour an area of skin over at least a portion of the femoral artery, the second flexible pad sized to contour around at least a portion of the user's inner thigh and outer thigh, the third flexible pad configured to contour an area of skin over at least a portion of a perineum, the third flexible pad sized to contour around at least one of a group consisting of at least a portion of the user's buttocks and at least a portion of the user's perineum; and a controller having processing circuitry in communication with the plurality of light sources, the processing circuitry configured to cause the plurality of light sources to emit a predetermined amount of light having at least two wavelengths from the plurality of light sources for at least one minute to increase, decrease or modulate sleep related hormone production in the target region.

In one embodiment, a flexible pad is configured to at least partially wrap around an area of skin over a neck of a mammal, the flexible pad having first, second, and third discrete portions having a plurality of light sources recessed within a corresponding one of the first, second, and third portions, the first portion being configured to be positioned on a first side of the mammal's neck, the second portion being configured to be positioned on a second side of the mammal's neck opposite the first side, and the third portion being configured to be positioned on a back side of the user's neck proximate skin over the cerebellum. A controller having processing circuitry is in communication with the plurality of light sources, the processing circuitry is configured to cause the plurality of light sources to emit a predetermined amount of light having at least four wavelengths from the plurality of light sources for at least one minute.

In one embodiment, an apparatus for promoting sexual wellness includes at least one flexible pad configured to contour an area of skin over a perineum region, the at least one flexible pad having a plurality of light sources, the at least one flexible pad including a section sized and configured to contour a user's thighs and a second section sized and configured to contour a female's genitals or a male's perineum.

In one embodiment the plurality of light sources is disposed on the second section.

In one embodiment the plurality of light sources is further disposed on the first section, and wherein the first section includes a first portion and an opposite section portion, and wherein the second section is disposed between the first portion and the second portion.

In one embodiment, the plurality of light sources is configured to emit at least four wavelengths of light.

In one embodiment, a method of promoting sexual wellness includes positioning at least one flexible pad over a perineum and emitting light for a predetermined period of time from a plurality of light sources disposed on the flexible pad.

In one embodiment, an apparatus for promoting sexual wellness includes a flexible pad configured to contour an area of skin over a perineum region, the at least flexible pad having a plurality of light sources, the flexible pad including a first section sized and configured to contour a user's thighs and a second section sized and configured to contour a user's genitals, the second section including the plurality of light sources, the plurality of light sources being configured to emit light at four wavelengths of length between 630-670 nm, 720-760 nm, 790-830 nm and 820-870 nm.

In one embodiment, an apparatus for promoting sleep includes a flexible pad including a first portion having a first plurality of light sources; a second portion having a second plurality of light sources, the second portion being opposite the first portion; and a third portion disposed between the first portion and the second portion, the third portion having a third plurality of light sources.

In one embodiment, the first portion, second portion, and third portion are discrete portions on the flexible pad.

In one embodiment, the first portion, second portion, and third portion are disposed at oblique angles with respect to each other.

In one embodiment, the first portion and third portion are intersected by a common longitudinal axis, and wherein the second portion is intersected by a longitudinal axis transverse to the common longitudinal axis.

In one embodiment, the plurality of light sources has four discrete wavelengths of light.

In one embodiment, the flexible pad defines a substantially closed loop.

### BRIEF DESCRIPTION OF THE DRAWINGS

A more complete understanding of embodiments described herein, and the attendant advantages and features thereof, will be more readily understood by reference to the following detailed description when considered in conjunction with the accompanying drawings wherein:

FIG. 1 shows an exemplary apparatus in accordance with the present disclosure, the apparatus including a control unit and a plurality of flexible pads;

FIG. 2 shows another exemplary apparatus constructed in accordance with the present disclosure in which the apparatus includes a plurality of cushions;

FIG. 3 shows another exemplary apparatus constructed in accordance with the present disclosure in which the apparatus is a strap;

FIG. 4 shows a plurality of flexible pads positioned in contact with different parts of a user's body;

FIG. 5 shows a flexible pad positioned in contact with an area of skin over a user's carotid artery;

FIG. 6A shows an exemplary apparatus for use over a user's perineum;

FIG. 6B shows the apparatus shown in FIG. 6A positioned in contact with an area of skin over a user's perineum and a user's buttocks;

FIG. 7 shows a flexible pad positioned in contact with an area of skin over the femoral artery and a flexible pad positioned in close proximity to the area of skin over the perineum; and

FIG. 8 shows a flow chart of an exemplary method for promoting sleep using the apparatus.

### DETAILED DESCRIPTION

Before describing in detail exemplary embodiments, it is noted that the embodiments reside primarily in combinations of apparatus components and processing steps related to promoting sleep. Accordingly, the apparatus and method components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

The terminology used herein is for the purpose of describing particular embodiments only and is not intended to be limiting of the concepts described herein. As used herein, the singular forms “a”, “an” and “the” are intended to include the plural forms as well, unless the context clearly indicates otherwise. It will be further understood that the terms “comprises,” “comprising,” “includes” and/or “including” when used herein, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this disclosure belongs. It will be further understood that terms used herein should be interpreted as having a meaning that is consistent with their meaning in the context of this specification and the relevant art and will not be interpreted in an idealized or overly formal sense unless expressly so defined herein.

As used herein, relational terms, such as “first” and “second,” “top” and “bottom,” and the like, may be used solely to distinguish one entity or element from another entity or element without necessarily requiring or implying any physical or logical relationship or order between such entities or elements. In embodiments described herein, the joining term, “in communication with” and the like, may be used to indicate electrical or data communication, which may be accomplished by physical contact, induction, electromagnetic radiation, radio signaling, infrared signaling or optical signaling, for example. One having ordinary skill in the art will appreciate that multiple components may interoperate and modifications and variations are possible of achieving the electrical and data communication.

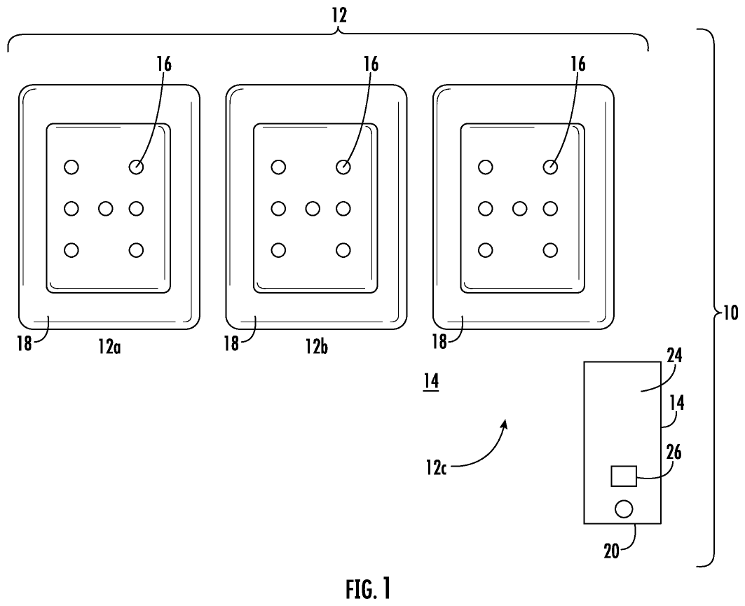

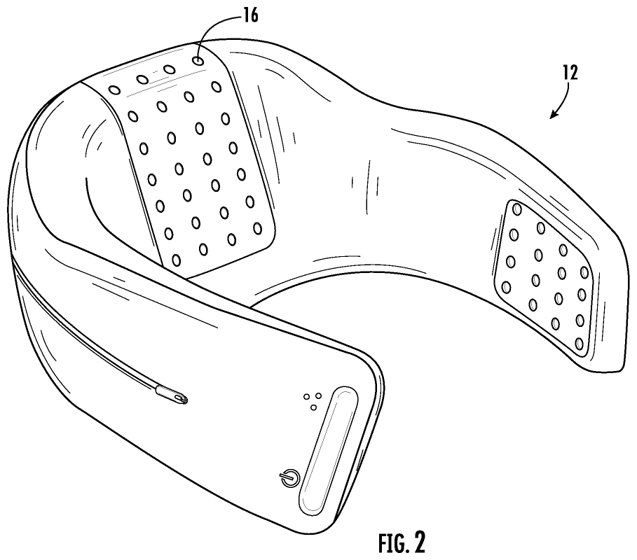

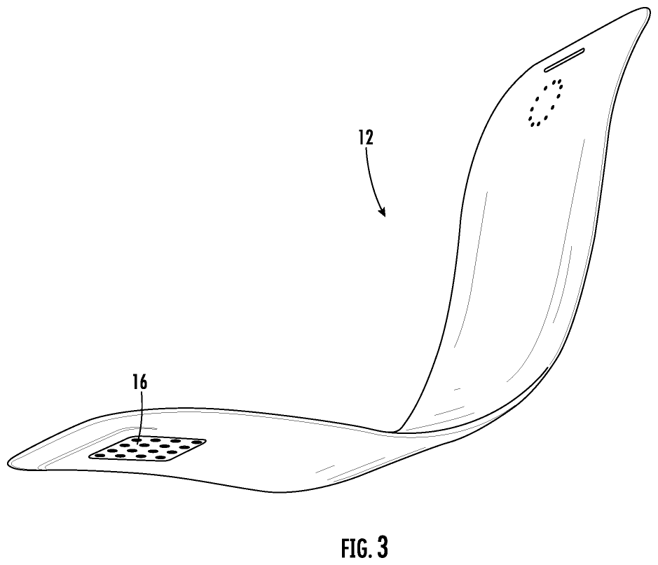

Referring now to FIG. 1, an exemplary apparatus 10 for promoting sleeping is shown. In one embodiment, the apparatus 10 generally includes at least one flexible pad 12 and a control unit/controller 14 in communication with the at least one flexible pad 12. In one embodiment, the apparatus 10 includes three flexible pads 12a, 12b, and 12c. Additionally, while the apparatus 10 is described in one embodiment as having three flexible pads 12, it will be understood that the apparatus may include one or more flexible pads 12. The flexible pads 12 may be composed of neoprene or polyester or similar material, and optionally may include a plurality of cushioning elements (FIG. 2) either contiguous along at least a portion of the respective flexible pad 12 or spaced apart from an adjacent cushioning element to contour a user's neck. Alternatively, the flexible pad may be an elongated strap (FIG. 3).

Continuing to refer to FIG. 1, in one embodiment, the apparatus 10 includes three flexible pads 12a, 12b, and 12c. Each flexible pad 12 may include one or more than one light source 16 and the light sources 16 may emit one wavelength of light and/or more than one wavelength of light. For example, the light sources 16 may be configured to emit a plurality of wavelengths of lights, for example, up to four wavelengths of light to irradiate tissue. In one configuration, the wavelengths of light include 650, 740, 810 and 850 nm. Each flexible pad 12 may have a customized size, shape, and number of light sources 16 and include one light source 16 or more than one light source 16. The light sources 16 may include light emitting diodes (“LEDs”), including but not limited to surface mount LEDs (“SMD LEDs”), bi-color LEDs, Red-Blue-Green LEDs (“RGB LEDs”) and high-power LEDs, or laser diodes. The plurality of light sources 16 may include light emitting diodes that emit a plurality of wavelengths of light having wavelengths of between 300 nanometers and 1300 nanometers which include red, near infrared, and infrared wavelengths of light. Each light source 16 may disposed within the flexible pad 12 so that the surface of the flexible pad 12 is smooth and can be placed against the skin without causing irritation. The flexible pad 12 and the light sources 16 may be disposed inside a housing 18 which may be designed based upon a variety of different human factors and ergonomics.

Light energy in the visible red (VR), infrared (IR) and near infrared (NIR) wavelengths may be beneficial for cellular metabolism. Light in these wavelengths is minimally dissipated as heat and is mainly absorbed by certain cellular chromophores. Light absorption in chromophores within the cellular respiratory chain (specifically—cytochrome c oxidase) leads to increased production of adenosine triphosphate (ATP) and reduction of reactive oxygen species (ROS). These increases in ATP lead to increased cellular metabolism. Reduction of ROS levels leads to an increase cellular proliferation and reduction of cellular apoptosis. These specific wavelengths may have a positive effect on the reduction of inflammation and increases in vascularization/blood flow.

Blood flow, inflammation and cellular metabolism are essential components of sexual wellness. PBM stimulation of these key metabolic actions have a significant positive effect on sexual wellness. NIR light may augment the release of dopamine, serotonin and endorphins responsible for sexual excitement and overall mood improvements

Continuing to refer to FIG. 1, in some embodiments, the control unit 14, which may be remote from the flexible pad 12 or integrated therein or thereon, also includes an energy generator (not shown). The energy generator may be a portable battery source that may be rechargeable, for example an external 120-240 VAC to 5-24 VDC power transformer/adapter and/or may include a power outlet, such as a 120 Volt power outlet. The control unit 14 may also include one or more controllers, processors, and/or software modules containing instructions or algorithms to provide for the automated or semi-automated operation and performance of the features, sequences, or procedures described herein. In one embodiment, the control unit 14 includes a computer 20 having a display 24 and processing circuitry 26 programmed or programmable to execute the automated or semi-automated operation and performance of the features, sequences, calculations, and/or procedures described herein. In one embodiment, the processing circuitry 26 includes a memory and a processor, the memory including instructions that, when executed by the processor, configured the processor to receive, process, or otherwise use signals from the light sources 16 and/or other system components. Additionally, in some embodiments the control unit 14 further includes one or more user input devices (such as a keyboard, touchscreen, keypad, button, knob, or the like), controllers, speakers, and/or displays that are in communication with processing circuitry 26 and used for collecting and conveying information from and to the user. For example, the control unit 14 and the processing circuitry 26 may be configured to manage the functional parameters of the apparatus 10 including the ability to turn the apparatus 10 on and off, to change the intensity and the frequency of the wavelengths of the light, to designate an irradiation time, to designate a frequency in cycles per second, to include audible alarms, and to charge the power source when the power source requires charging based upon preset parameters that are programmed into the processing circuitry 26.

Continuing to refer to FIG. 1, each flexible pad 12 may be in communication with the control unit 14 and may be physically and integrally connected to the control unit 14 with, for example, a wire. Alternatively, each flexible pad 12 may be wireless and in communication with the control unit 14. The processing circuitry 26 within the control unit 14 may be programmed to provide a set amount of irradiation through each flexible pad 12 and light source 16. For example, each flexible pad 12 and light source 16 may provide the same amount of irradiation or each flexible pad and light source may provide different amounts of irradiation. The processing circuitry 26 may also be programmed to provide irradiation for a predetermined period of time. The predetermined period of time may be set for any period of time, for example, for at least one minute. Alternatively, the predetermined period of time may be more or less time depending upon the needs and requirements of the particular user that is using the apparatus 10. Some individuals may require longer irradiation times while others may require shorter irradiation times and the processing circuitry 26 may be set to a variety of different times. Each flexible pad 12 may be set to a different predetermined period of time, certain flexible pads 12 may be set for a particular period of time while others may be set to a different period of time, or each flexible pad 12 may be set for different predetermined periods of time. The predetermined period of time may be configured based upon the amount of irradiation required to increase, decrease or modulate the sleep related hormone production in any particular area of the body.

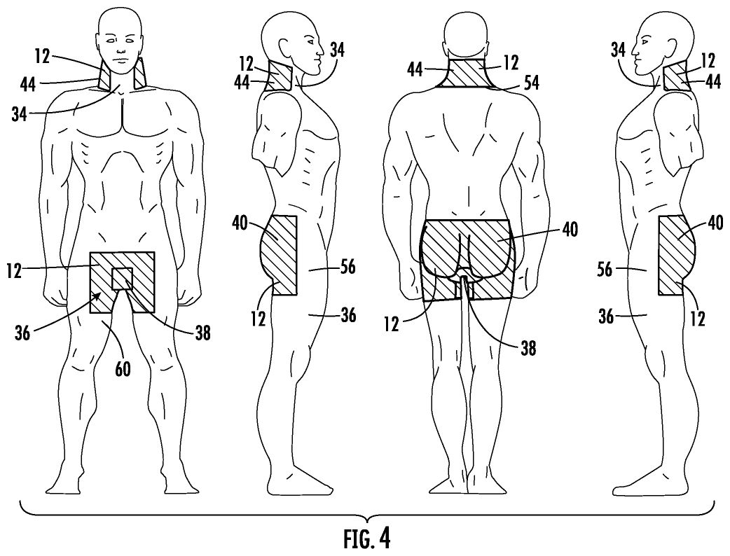

Referring now to FIGS. 4-6, each flexible pad 12 may be configured to contour an area of skin over a target region within the body. The target region may be anywhere on the body and may include a large or small portion of the body. As shown in FIG. 4, in one embodiment the target region may include an area of skin over the carotid artery 34, an area of skin over the femoral artery 36, an area of skin over the perineum 38, and an area of skin over the buttocks 40. The size and shape of the flexible pad 12 may be based upon the size and shape of the particular target region as well as the individual that is using the apparatus 10. The flexible pad 12 may be secured to and/or around any particular target region with a securing mechanism. The securing mechanism may include an adhesive, a tab configured to wrap around at least a portion of the body, a hook and loop fastener, and/or the flexible pad 12 may be made from a material that is movable from a first position to a second position and can remain securely in the first position and/or the second position. Alternatively, the flexible pad 12 may include a metallic or non-metallic strip or other type of rigid and/or semi rigid strip or structure that is configured to be secured around at least a portion of the body.

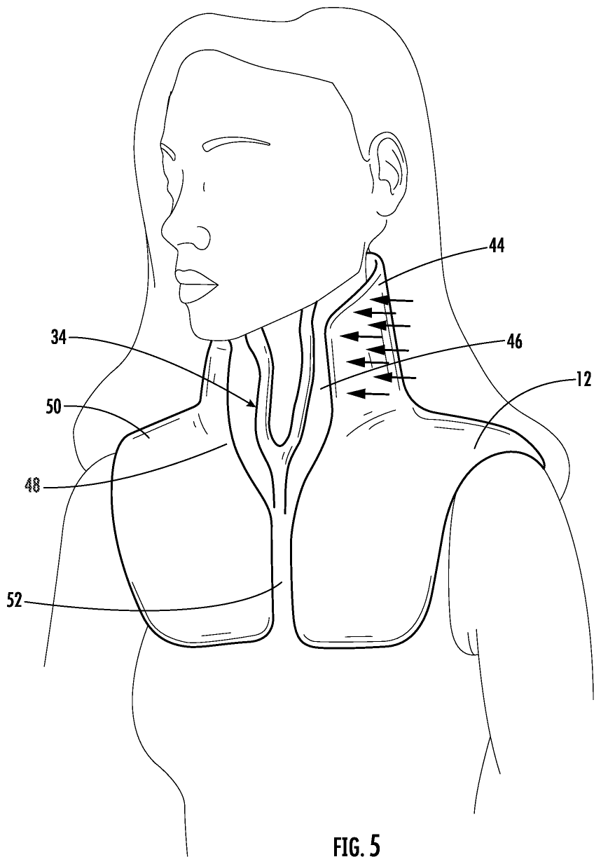

Referring now to FIG. 5, the flexible pad 12 may be configured to contour the area of skin over the carotid artery 34. In this exemplary embodiment, the flexible pad 12 may include more than one portion. For example, the flexible pad 12 may include a first portion 44 and the first portion 44 may be sized and shaped to contour an area of skin around at least a portion of the neck 46. The first portion 44 may be sized and shaped to contour around the entire neck or the first portion 44 may be configured to contour around only a portion of the neck. As shown in FIG. 5, the first portion 44 is contoured around only a portion of the skin around the neck 46 while the portion of the skin on the neck below the chin of the user remains unrestricted by the flexible pad 12. The flexible pad 12 may also include a second portion 48 and the second portion may be sized and shaped to contour around at least a portion of the shoulders 50, the upper chest 52, and the upper back 54 (shown in FIG. 4). As shown in FIG. 5, the second portion 48 of the flexible pad 12 may only contour around a portion of each shoulder 50 so that each arm can freely rotate and move while the second portion 48 of the flexible pad 12 is contoured around a portion of the shoulders 50. The flexible pad 12 configured to contour the area of skin over the carotid artery 34 may include a plurality of light sources 16 on only a portion of the flexible pad 12 that is in direct contact with the skin or the plurality of light sources 16 may be arranged in any manner or fashion on the portion of the flexible pad 12 that is in direct contact with the skin.

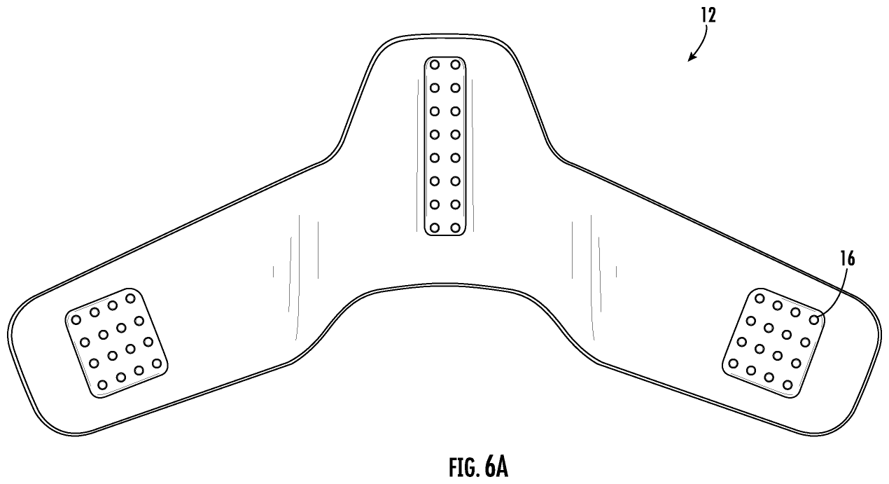

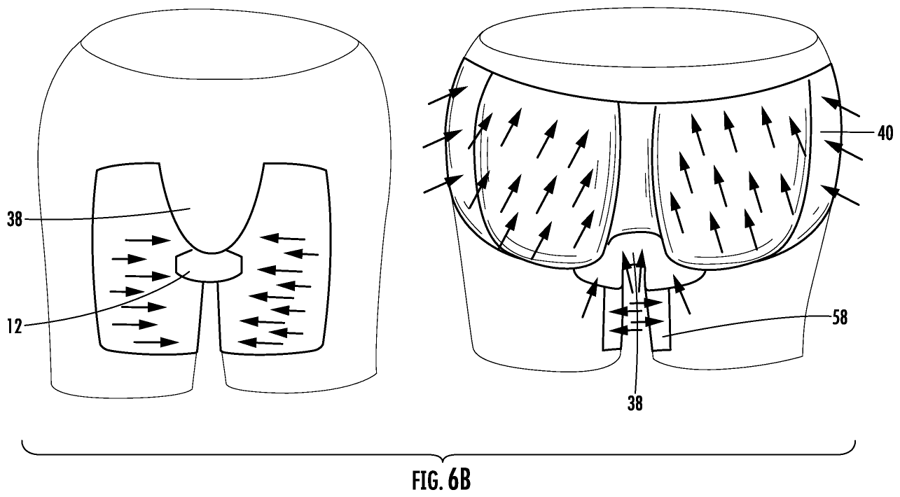

Referring now to FIGS. 6A-6B, the flexible pad 12 may be configured to contour the area of skin over the perineum 38 and the genitals to promote sexual wellness. For example, as shown in FIG. 6B, the flexible pad 12 may contour a portion of the skin over the perineum 38 as well as an area of skin over the buttocks 40 of the user. In one example, the flexible pad 12 may only contour a portion of the skin over the perineum 38 and may also be configured to contour a portion of the skin over the upper thigh 56 (as shown in FIG. 4). In another example, the flexible pad 12 may only contour a portion of the skin over the buttocks 40, a portion of the skin over the inner thigh 58, as well as a portion of the skin over the perineum 38. Alternatively, the flexible pad 12 may be configured to contour around the entire waist of the user including the area of skin over the buttocks 40, the area of skin over the perineum 38, the area of skin over the inner thigh 58, and the area of skin over the upper thigh 56. The flexible pad 12 may be configured in a variety of different sizes and shapes to contour the anatomy of the user from the lower waist down to the upper thighs. Additionally, the number of light sources 16 may be configured in a variety of different manners to provide irradiation to different areas of the body with various wavelengths. For example, the light sources may be positioned on three distinct sections of the flexible pad 12 to irradiate the genitals and the inner thigh area.

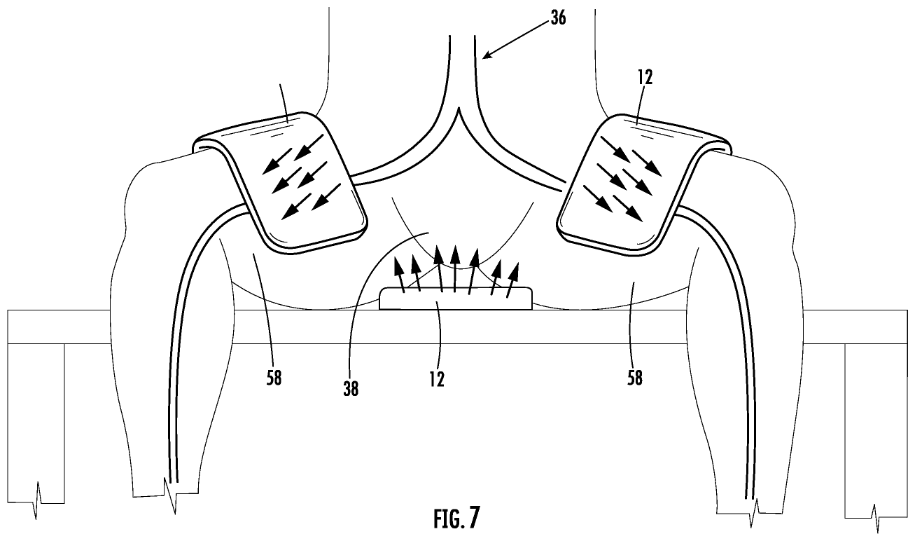

Now referring to FIG. 7, the flexible pad 12 may also be configured to contour the area of skin over the femoral artery 36. At least one flexible pad 12 may be configured to contour at least a portion of the skin on the inner thigh 58 and at least a portion of the skin on the outer thigh 60 (as shown in FIG. 4). The thickness of the flexible pad 12 may be based upon the anatomy of the user including the size of the thigh. The portion of the flexible pad 12 that is in direct contact with the skin of the user may include a plurality of light sources 16 configured to provide light to the portion of the body that the light is in contact with. The flexible pad 12 may be configured to contour around the entire circumference of the user's thigh, or it may be configured to contour around only a portion of the circumference of the user's thigh. Also, as shown in FIG. 7, the area of skin over the perineum 38 may be irradiated with a flexible pad 12 that is below the area of skin over the perineum 38. For example, the flexible pad 12 may be placed on a surface such as a chair and/or bench, and the user may position the area of skin over the perineum 38 just above the flexible pad 12 and the light sources 16 may be on the portion of the flexible pad 12 that is facing toward the area of skin over the perineum 38. This may allow for the irradiation of the particular target region without having the flexible pad 12 in contact with the user's skin. This may be helpful where a user has sensitive skin or skin that is easily irritated while still allowing the area of skin to be irradiated.

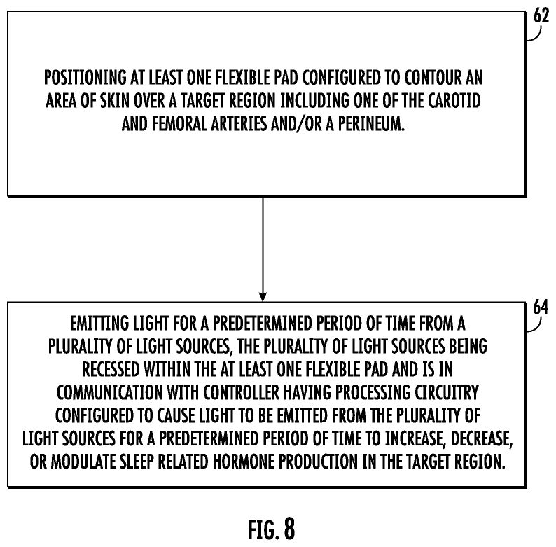

Now referring to FIG. 8, a flow chart of an exemplary method for promoting sleep using the apparatus 10 is shown. In a first step 62, the at least one flexible pad 12 may be positioned to contour an area of skin over the target region. The target region may include the area of skin over the carotid artery 34, the area of skin over the femoral artery 36, and the area of skin over the perineum 38. The flexible pad 12 may also be positioned to contour an area of skin over any other body part of the body of the user. Continuing to refer to FIG. 8, in a second step 64, light may be emitted for the predetermined period of time from the plurality of light sources 16. The plurality of light sources 16 may be recessed within the at least one flexible pad 12 and may be in communication with the control unit 14 having processing circuitry 26 being in communication with the processing circuitry 26 configured to emit light from the plurality of light sources 16 for the predetermined period of time to increase, decrease or modulate sleep related hormone production in the target region.

In other configurations, the flexible pad 12 may be configured to contour skin over a female's genitals or surrounding area and a male's prostate or surrounding area. In such a configuration, the plurality of light sources 16 may irradiate the genitals in the manner described above and improve or promote sexual function and wellness in both men and women.

It will be appreciated by persons skilled in the art that the present embodiments are not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. An apparatus for promoting sleep, comprising:
a flexible pad including:
a first portion having a first plurality of light sources;
a second portion having a second plurality of light sources, the second portion being opposite the first portion; and
a third portion disposed between the first portion and the second portion, the third portion having a third plurality of light sources.

29. The apparatus of claim 28, wherein the first portion, second portion, and third portion are discrete portions on the flexible pad.

30. The apparatus of claim 29, wherein the first portion, second portion, and third portion are disposed at oblique angles with respect to each other.

31. The apparatus of claim 29, wherein the first portion and third portion are intersected by a common longitudinal axis, and wherein the second portion is intersected by a longitudinal axis transverse to the common longitudinal axis.

32. The apparatus of claim 28, wherein the plurality of light sources has four discrete wavelengths of light.

33. The apparatus of claim 28, wherein the flexible pad defines a substantially closed loop.

34. The apparatus of claim 32, wherein the discrete wavelengths of light are in the range of 630-670 nm, 720-760 nm, 790-830 nm and 820-870 nm.

35. The apparatus of claim 28, wherein the plurality of light sources is configured to emit light having four wavelengths of light between 300-1300 nanometers.
